(19) United States
(12) Patent Application Publication
(10) Pub. No.: US 2026/0088155 A1
(43) Pub. Date: Mar. 26, 2026

(54) METHOD AND SYSTEM FOR PROCESSING TISSUE SECTION IMAGES

(71) Applicant: Chengdu Wision Medical Device Co., Ltd., Chengdu (CN)

(72) Inventors: XIAO XIAO, CHENGDU (CN); QIANG GE, CHENGDU (CN); XIAOHAI XU, CHENGDU (CN); JINGJIA LIU, CHENGDU (CN)

(21) Appl. No.: 19/023,316

(22) Filed: Jan. 16, 2025

(30) Foreign Application Priority Data
Sep. 24, 2024 (CN) 202411334426.0

(51) Int. Cl. G16H 30/40; G06T 3/147; G06T 7/00; G06T 7/564; G06T 7/70

(52) U.S. Cl. CPC G16H 30/40; G06T 3/147; G06T 7/0012; G06T 7/564; G06T 7/70; G06T 2207/30024; G06T 2207/30204

(57) ABSTRACT

A method for processing tissue section images is provided. The method includes: (S1) providing a paraffin-embedded tissue block; (S2) determining at least three first coordinates on tissue sections to be sliced off from the paraffin-embedded tissue block, ablating at the first coordinates to form marker points on the tissue sections, and slicing the ablated tissue sections off from the paraffin-embedded tissue block; (S3) acquiring a slide image of each of the ablated tissue sections; and (S4) determining a second coordinate corresponding to each marker point on the slide image, comparing for each marker point the second coordinate with the first coordinate, and processing the slide image according to the comparison results. A system for performing the method for processing tissue section images is also provided.

### CROSS-REFERENCE TO RELATED APPLICATION

This application claims priority under 35 U.S.C. § 119 to Chinese Patent Application No. 202411334426.0, filed on Sep. 24, 2024, the entirety of which is incorporated herein by reference.

### FIELD OF THE INVENTION

The present disclosure relates to artificial intelligence in medicine, and more particularly to a computer-implemented method and an artificial intelligent system for processing tissue section images to be used in three-dimensional reconstruction.

### BACKGROUND OF THE INVENTION

In recent years, three-dimensional (3D) reconstruction technology has been widely applied in clinical diagnosis and treatment in fields such as hepatobiliary surgery, plastic surgery, maxillofacial surgery, and orthopedics. 3D reconstruction technology provides comprehensive, stereoscopic, and intelligent dynamic images or static images, clearly displays the spatial location and morphological structure of a lesion, and anatomical relationships of the lesion with its surrounding organs, and is helpful for preoperative planning, measurement, calculation, simulated surgical operations, formulation of individualized treatment plans, and improving surgical accuracy and safety.

In the process of 3D reconstruction of tissues, after performing a series of processing steps on the histopathological slide images, positioning of each slide image is required. The precision of this positioning step directly affects the accuracy of 3D reconstruction of tissues. The inventors' team disclosed a method for 3D reconstruction of a tumor-affected tissue in patent publication CN117011462A. In that method, after histopathological slide images are obtained by using a tissue scanner, three marks are made on the slide images. The slide images are then rotated and/or flipped based on the positional relationships of the three marks to ensure the angles and sides of the slide images are properly aligned, thereby achieving accurate positioning of the slide images.

In order to obtain more sectional information of a tissue for a more precise 3D reconstruction, histopathological sections need to be as thin as possible (e.g., 2 μm), which in turn causes significant increases in the number of slide images that requires to be positioned. However, as described above, the existing method for positioning slide images involves generating three marks on each slide image according to a series of algorithms; consequently, when the number of slide images increases, the efficiency of image positioning would reduce. Moreover, since the marks used to align the slide images are not based on the same coordinate system, the slide images can only be aligned according to the positional relationships among the three marks, which inevitably introduces errors. When the number of the slide images is large, cumulative errors can severely affect positioning precision, further impacting the precision of 3D reconstruction. Therefore, efficiency and precision of the existing slide image positioning method is inadequate for large-scale slide image processing, and precision of 3D tissue reconstruction needs further improvement.

### BRIEF SUMMARY OF THE INVENTION

One object of the present disclosure is to provide a method for processing tissue section images used in 3D tissue reconstruction, aiming to address issues of low efficiency, significant cumulative errors, and poor accuracy in processing large-scale slide images, and to enable efficient and high-precision positioning of slide images, thus allowing acquisition of a larger number of thinner tissue sections during the tissue slicing process, thereby significantly improving precision of 3D tissue reconstruction.

The above-mentioned object is achieved by the following technical solutions:

A method for processing tissue section images is provided. The method includes the steps of:
(S1) providing a paraffin-embedded tissue block;
(S2) determining at least three first coordinates on one or more tissue sections to be sliced off from the paraffin-embedded tissue block, ablating at the first coordinates to form marker points on the tissue sections, and slicing the ablated tissue sections off from the paraffin-embedded tissue block;
(S3) acquiring a slide image of each of the ablated tissue sections; and
(S4) determining a second coordinate corresponding to each of the marker points on the slide image, comparing for each of the marker points the second coordinate with the first coordinate, and processing the slide image according to the comparison results.

In the present technical solution, a tissue of interest is first embedded in paraffin to form a paraffin-embedded tissue block. In one or more embodiments, the paraffin-embedded tissue block may be pretreated, for example, by trimming off excess paraffin until the tissue is exposed on an end face of the paraffin-embedded tissue block.

In the present technical solution, the paraffin-embedded tissue block is sliced into a plurality of tissue sections by using a microtome. During the slicing process, a laser is used to ablate several marker points on each tissue section. In some embodiments, the laser ablates an end face or a cut cross-section of the paraffin-embedded tissue block to form one or more marker points thereon; by slicing the ablated end face or cut cross-section off from the paraffin-embedded tissue block, tissue sections with one or more marker points are obtained. In one or more embodiments, depth of the laser ablation, i.e., the length in a direction perpendicular to each cross-section of the tissue, is an integer multiple of a thickness of one tissue section, thereby improving efficiency of the ablation. In some preferred embodiments, the depth of laser ablation equals the thickness of one tissue section, so as to ensure that the marker points consistently fall within a preferred region (e.g., the tissue contour) across every tissue section.

In the present technical solution, the laser or its emission device is movable, allowing the positions of the ablated marker points to vary from one tissue section to another. The laser of the controller thereof is equipped with a two-dimensional coordinate system, allowing the laser or its emission device to move to first coordinates based on the two-dimensional coordinate system. When the laser ablates at the first coordinates to obtain the marker points, the first coordinates for each tissue section are recorded.

After forming marker points on each tissue section, a tissue scanner may be used to scan the tissue sections, thereby obtaining slide images corresponding to each tissue section. The slide images sequentially numbered according to the slicing order. In some embodiments, additional operations may be performed on each slide image, such as defining one or more closed infiltration zones as described in CN117011462A.

In the present technical solution, a second coordinate corresponding to each of the marker points on the tissue section in the slide image is determined. The second coordinate system used for the second coordinates may be the same as or different from the first coordinate system used for the first coordinates. In some embodiments, the first and second coordinate systems are identical, so that the second coordinate can be compared directly to the first coordinate. However, in some embodiments where the first and second coordinate systems are different, the second coordinates in the second coordinate system are converted into the first coordinate system through a spatial relationship between these two coordinate systems, after which the converted second coordinates are compared with the first coordinates. By comparing the second coordinates on the slide image one by one with the recorded first coordinates, the second coordinates are moved to their corresponding or nearest first coordinates, thereby processing (e.g., rotating and/or flipping) the slide image to achieve precise positioning of the slide image.

In the present technical solution, a plurality of marker points is ablated directly on each tissue section by a laser, thereby ensuring high marking efficiency and precision. More importantly, since the first coordinates of the marker points on different tissue sections share the same two-dimensional coordinate system, after the second coordinates of the marker points on each slide image are determined, comparing the second coordinates with the corresponding first coordinates allows the slide image to be rotated and/or flipped based on the same two-dimensional coordinate system. Therefore, all the slide images are properly aligned and original positions of the tissue sections are restored, thereby significantly reducing cumulative errors, improving positioning efficiency, facilitating acquisition of a large number of slide images of thin tissue sections during tissue slicing, and markedly enhancing the precision of 3D reconstruction of tissue samples.

Furthermore, the step of (S2) includes steps of:
acquiring a cross-sectional image of the tissue section;
extracting a tissue contour from the cross-sectional image;
determining within the tissue contour the first coordinates;
ablating the tissue section at the first coordinates to form the marker points thereon; and
slicing the tissue section off to obtain the ablated tissue section.

In the present technical solution, prior to slicing each tissue section off from the paraffin-embedded tissue block, a camera can be used to capture a cross-sectional image of the tissue section to be sliced. Next, a tissue contour (i.e., a boundary between the tissue and paraffin) is extracted from the cross-sectional image.

Subsequently, the first coordinates of the marker points are determined within the extracted tissue contour. These first coordinates may be distributed randomly or have certain spatial relationships. For example, the geometric center of a polygon formed by lines connecting the first coordinates may coincide with the centroid of the tissue contour. In some preferred embodiments, the first coordinates fall within a vicinity of the center of the tissue section. This is because, compared to peripheral areas of the tissue section, areas closer to the center is less likely to deform. Therefore, locating the first coordinates at the vicinity of the center of the tissue section may minimize shifts or deformation of the marker points during slicing.

In the present technical solution, the laser ablates a certain depth at the determined first coordinates. The depth of the laser ablation preferably equals the thickness of the tissue section, or slightly less than the thickness of the tissue section, so as to allow the location of the marker points to vary section by section. After forming the marker points, the tissue section is sliced off from the paraffin-embedded tissue block, resulting in a tissue section ablated with the marker points.

In the present technical solution, the first coordinates are selected based on the tissue contour to ensure that all marker points are located on the tissue, thereby overcoming the issue that marker points ablated on the paraffin cannot be captured in the slide image due to melting of the paraffin before slide image acquisition.

Furthermore, the first coordinates are determined according to the following steps: determining a centroid of the tissue contour; and generating the first coordinates around the centroid, wherein a geometric center of a polygon formed by lines connecting the first coordinates falls within a vicinity of the centroid.

In the present technical solution, the first coordinates of the marker points on any given tissue section have certain spatial relationships. Specifically, the centroid of the tissue contour is calculated based on coordinates of several points along the tissue contour. The centroid Xₘ is calculated using the formula:

$${X}_{m} = \frac{\sum {m}_{i} {x}_{i}}{\sum {m}_{i}} ,$$

wherein xᵢ refers to a coordinate of any point on the tissue contour, and mi refers to a weight of that point. In some embodiments, all points on the tissue contour are assigned equal weights.

In the present technical solution, at least three first coordinates may be randomly generated within the tissue contour. The first coordinates are connected to form a polygon, whose vertices are located at the first coordinates. The geometric center of the polygon is located close to the centroid of the tissue contour. In some embodiments, the geometric center of the polygon may coincide with the centroid.

By locating the geometric center of the polygon formed by the marker points within the vicinity of the centroid of the tissue contour, the marker points are evenly distributed across the tissue section, avoiding severe local damage to the tissue section caused by clustering of marker points.

Some preferred embodiments of the present disclosure for determining the first coordinates further includes the following steps:
for a first tissue section of the paraffin-embedded tissue block, determining a first centroid of a first tissue contour of the first tissue section, and generating the first coordinates around the first centroid, wherein a geometric center of a first polygon formed by lines connecting the first coordinates coincides with the first centroid; and
for each of the subsequent tissue sections, obtaining the first coordinates of the marker points on the preceding tissue section, shifting the first coordinates toward a same direction and for a same distance, determining whether all of the shifted first coordinates fall within a second tissue contour of the current tissue section, and
if all the shifted first coordinates fall within in the second tissue contour, defining the shifted first coordinates as the first coordinates of the marker points to be ablated on the current tissue section; or
if at least one of the shifted first coordinates fall outside of the second tissue contour, determining a second centroid of the second tissue contour, relocating the shifted first coordinates so that a geometric center of a second polygon formed by lines connecting the relocated first coordinates coincides with the second centroid, and defining coordinates corresponding to vertices of the second polygon as the first coordinates of the marker points to be ablated on the current tissue section.

In such technical solution, for the first tissue section of the paraffin-embedded tissue block, the first coordinates are randomly generated within the tissue contour of the first tissue section. A geometric center of a polygon formed by lines connecting the first coordinates coincides with the centroid of the tissue contour of the first tissue section.

In such technical solution, for the second and subsequent tissue sections of the paraffin-embedded tissue block, the first coordinates for the preceding tissue section is obtained first and then used to determine the first coordinates for the current tissue section. For example, for the second tissue section of the paraffin-embedded tissue block, the first coordinates of the first tissue section are acquired, and then the acquired first coordinates are shifted toward a same direction and for a same distance to obtain the shifted first coordinates.

Subsequently, each of the shifted first coordinates is acquired to determine whether it falls within the tissue contour of the second tissue section or not. If all the shifted first coordinates fall within the tissue contour of the second tissue section, the shifted first coordinates are defined as the first coordinates of the marker points to be ablated on the second tissue section. On the contrary, if at least one of the shifted first coordinates fall outside of the tissue contour of the second tissue section, a second centroid of the tissue contour of the second tissue section is acquired, the shifted first coordinates are relocated so that a geometric center of a second polygon formed by lines connecting the relocated first coordinates coincides with the second centroid, and the coordinates corresponding to vertices of the second polygon are defined as the first coordinates of the marker points to be ablated on the second tissue section.

In the present technical solution, the first coordinates for the subsequent tissue section are selected according to the first coordinates on the preceding tissue section, so as to ensure that the first coordinates on adjacent tissue sections do not overlap, thereby effectively reducing the likelihood of laser ablation to affect the cancerous area on the tissue section. For example, if one first coordinate on the first tissue section falls within a cancerous area, that first coordinate will be shifted away and not become the first coordinate on the second tissue section; consequently, the same coordinate on the second tissue section will not be ablated again, thereby effectively reducing damage to the cancerous area by the laser.

Furthermore, if at least one of the shifted first coordinates falls outside of the second tissue contour, the method further includes a step of: after relocating the shifted first coordinates, rotating the second polygon formed by the lines connecting the relocated first coordinates.

In some preferred embodiments, for each of the subsequent tissue sections, the first coordinates are determined according to the following step: if the first coordinates of the marker points on the preceding tissue section are shifted toward a first direction and if all of the shifted first coordinates are determined to fall within the second tissue contour, further shifting the shifted first coordinates in the first direction to generate the first coordinates of the marker points to be ablated on the current tissue section.

In order to make shifting of the first coordinates on adjacent tissue sections more consistent and thus to avoid cancerous areas, in this technical solution, if all of the first coordinates of the preceding tissue section, after being shifted in the first direction, are still within the tissue contour of the current tissue section, the first coordinates of the subsequent tissue section continue to be shifted in the same first direction.

For example, after shifting the first coordinates of the first tissue section in the first direction, if the shifted first coordinates are still within the tissue contour of the second tissue section, the shifted first coordinates is used as the first coordinates of the second tissue section. Next, when determining the first coordinates of the third tissue section, the first coordinates of the second tissue section would continue to be shifted in the first direction. Such process repeats until, when determining the first coordinates of a specific tissue section, the shifted first coordinates lie outside the tissue contour of that specific tissue section. In that case, the geometric center of the polygon formed by lines connecting the first coordinates is moved to the centroid of the tissue contour of that specific tissue section.

Furthermore, the step of (S2) includes a step of: prior to slicing a tissue section off from the paraffin-embedded tissue block, adhering a film onto the tissue section.

Since tissue sections being sliced off from the paraffin-embedded tissue block are very thin, they may more or less deform or curl during the slicing process, thus making it difficult to extract the accurate locations of the marker points from the slide images.

In the present technical solution, applying a thin film to the tissue section can effectively increase the strength of the tissue section, thereby reducing deformation, curling, and wrinkling of the tissue section during the slicing process, and lowering the likelihood of displacement of the marker points in the subsequent steps. In some embodiments, the film can be applied to the tissue section before the marker points are ablated on the tissue section. In one or more embodiments, the tissue section may be ablated with the marker points first, followed by adhesion of the film onto the ablated tissue section.

The step of (S4) further includes steps of:
for each of the marker points, comparing the first and second coordinates to obtain an affine transformation matrix;
applying the affine transformation matrix to the slide image to obtain a third coordinate for each of the marker points; and
processing the slide image according to comparison results between the third coordinates and the first coordinates.

During the slicing process, an external force from the microtome may cause the tissue section to stretch, rotate or otherwise deform, which may become more pronounced as the thickness of the tissue section decreases. Such deformation issue not only makes it difficult to align the second coordinates on the slide image with the corresponding first coordinates, resulting in rough positioning of the slide image according to the similarity between the first and second coordinates, but also affects the precision of subsequent 3D reconstruction.

To solve the aforementioned issue, in the present technical solution, after the second coordinates of the marker points on the slide image are determined, an affine transformation matrix corresponding to the slide image is obtained based on the first and second coordinates. The formula for calculating the affine transformation matrix is as follows:

$$[ \begin{matrix}{x}^{′} \\ {y}^{′} \\ 1\end{matrix} ] = [ \begin{matrix}{s}_{x} & {α}_{x} & {t}_{x} \\ {α}_{y} & {s}_{y} & {t}_{y} \\ 0 & 0 & 1\end{matrix} ] [ \begin{matrix}x \\ y \\ 1\end{matrix} ]$$

wherein x and y are the first coordinates (x, y), x′ and y′ are the second coordinates (x′, y′), and the six variables in the affine transformation matrix are as follows: sₓ and s_{y} respectively control the scaling and flipping along the X and Y axes, αₓ and α_{y} respectively control the rotation along the X and Y axes, and tₓ and t_{y} respectively control the translational motion along the X and Y axes.

Based on the first and second coordinates of the marker points on the tissue section and on the slide image respectively, the six variables in the affine transformation matrix can be obtained by solving a system of six linear equations. The affine transformation matrix is then applied to each pixel in the slide image to correct the slide image. Next, third coordinates of the marker points are identified in the corrected slide image; since the third coordinates aligned better with the first coordinates, more precise positioning of the slide image can be achieved. Additionally, using slide images corrected based on the first coordinates can result in better restoration of the shape of the tissue section, thereby improving the quality and precision of subsequent 3D reconstruction.

Furthermore, if one or more of the tissue sections is ablated with at least four marker points, the step of (S4) comprises steps of:
dividing a polygon formed by lines connecting the at least four marker points into at least two triangular regions;
obtaining the affine transformation matrix of each triangular region; and
for pixels of the slide image locating within one of the triangular regions, processing the slide image by applying to the pixels the affine transformation matrix corresponding to the triangular region at which the pixels locate; or
for other pixels of the slide image locating outside of any one of the triangular regions, processing the slide image by applying to the other pixels the affine transformation matrix corresponding to the triangular region closest to which the other pixels locate.

For tissue sections having three marker points, the first and second coordinates of these three marker points can be used to calculate the six variables of the affine transformation matrix, thus correcting the slide image. To further improve the correction accuracy, more marker points may be required.

In the present technical solution, the slide image includes at least four marker points thereon. The at least four marker points can form polygons such as quadrilaterals, pentagons, or hexagons. In that case, before solving the affine transformation matrix, the polygon is first divided into multiple triangular regions. For example, a quadrilateral is divided into two triangular regions, and a pentagon is divided into three triangular regions. Subsequently, the first and second coordinates of the three marker points on each triangular region are used to solve the affine transformation matrix corresponding to the triangular region. Next, for pixels of the slide image locating within one of the triangular regions, the slide image is processed by applying to the pixels the affine transformation matrix corresponding to the triangular region at which the pixels locate. Alternatively, for other pixels of the slide image locating outside of any one of the triangular regions, processing the slide image by applying to the other pixels the affine transformation matrix corresponding to the triangular region closest to which the other pixels locate, thereby completing the correction of the entire slide image.

In the present technical solution, more marker points lead to more triangular regions, allowing different affine transformation matrices to be applied to different triangular regions for image correction, thereby facilitating the correction of tissue section having more complex tissue deformations and further improving the precision of the image correction.

Furthermore, the step of (S4) further includes the steps of:
dividing the polygon in at least two ways to form at least two groups of triangular regions, and obtaining at least two groups of affine transformation matrices for each group of the triangular regions;
separately processing the slide image by applying the at least two groups of the affine transformation matrices to obtain at least two corrected tissue contours of the slide image; and
selecting the group of affine transformation matrices corresponding to the corrected tissue contour that has the highest resemblance to a cross-sectional image of a tissue section acquired before the tissue section is sliced off from the paraffin-embedded tissue block.

In the present technical solution, different ways are used to divide the polygon into several groups of triangular regions. For example, two diagonals of a quadrilateral can be connected to respectively divide the quadrilateral into a first group of triangular regions and a second group of triangular regions. Then, two first affine transformation matrices are calculated for the two triangular regions in the first group; and the two first affine transformation matrices are applied to the slide image to obtain a first corrected image. Similarly, two second affine transformation matrices are calculated for the two triangular regions in the second group, and the two second affine transformation matrices are applied to the slide image to obtain a second corrected image. Two corrected tissue contour corresponding to the first and second corrected slide images are extracted and compared with the tissue contour in the cross-sectional image of the tissue section. According to the comparison result, the corrected slide image that has the corrected tissue contour shape closest to the shape of the cross-sectional image is taken as the corrected slide image.

By using different ways for dividing the polygon and selecting the best division based on the tissue contour in the cross-sectional image, accuracy and precision of the slide image correction is further improved.

Another object of the present disclosure is to provide a system for processing tissue section images. The system includes:
a microtome for slicing a paraffin-embedded tissue block to obtain a plurality of tissue sections;
a computing device for determining at least three first coordinates on at least a portion of the tissue sections;
a laser for emitting laser beams to ablate at least three marker points at the first coordinates on each of the tissue sections;
a tissue scanner for acquiring a slide image of each of the ablated tissue sections; and
an image processor for determining a second coordinate corresponding to each of the marker points on the slide image, comparing for each of the marker points the second coordinate with the first coordinate, and processing the slide image according to the comparison results.

As compared with the prior art, the embodiments provided in the present disclosure has the following advantages and beneficial effects:
1. The methods provided in the present disclosure adopt a laser to directly ablate a plurality of marker points on each tissue section, so that high marking efficiency and precision is ensured. More importantly, since the first coordinates of the marker points on different tissue sections share the same two-dimensional coordinate system, after the second coordinates of the marker points on each slide image are determined, comparing the second coordinates with the corresponding first coordinates allows the slide image to be rotated and/or flipped based on the same two-dimensional coordinate system. Therefore, all the slide images can be properly aligned and original position of the tissue sections can be restored, thereby significantly reducing cumulative errors, improving positioning efficiency, facilitating acquisition of a large number of slide images of thin tissue sections during tissue slicing, and markedly enhancing the accuracy and precision of 3D tissue reconstruction.
2. The methods provided in the present disclosure select the first coordinates based on the tissue contour to ensure that all marker points are located on the tissue, thereby overcoming the issue that marker points ablated on the paraffin cannot be captured in the slide image due to melting of the paraffin before slide image acquisition.
3. The methods provided in the present disclosure locate the geometric center of the polygon formed by lines connecting the marker points within the vicinity of the centroid of the tissue contour, thereby allowing the marker points evenly distribute across the tissue section and avoiding severe local damage to the tissue section caused by clustering of marker points.
4. The methods provided in the present disclosure determine the first coordinates for the subsequent tissue section according to the first coordinates of the preceding tissue section, so as to ensure that the first coordinates on adjacent tissue sections do not overlap, thereby effectively reducing the likelihood of laser ablation to affect the cancerous area of the tissue section.
5. The methods provided in the present disclosure calculate the affine transformation matrix based on the first and second coordinates of the marker points on the tissue section and on the slide image respectively, thus making the third coordinates of the marker points in the corrected slide image to align better with the first coordinates, thereby achieving more accurate and precise positioning of the slide image. Additionally, using slide images corrected based on the first coordinates can result in better restoration of the shape of the tissue section, thereby improving the quality and precision of subsequent 3D reconstruction.
6. The methods provided in the present disclosure use more marker points to define more triangular regions, allowing different affine transformation matrices to be applied to different triangular regions for image correction, thereby facilitating the correction of tissue section having more complex tissue deformations and further improving the accuracy and precision of the image correction.

### BRIEF DESCRIPTION OF THE DRAWINGS

The present invention will become more readily apparent to those ordinarily skilled in the art after reviewing the following detailed description and accompanying drawings, in which:

FIG. 1 is a flowchart of a method for processing tissue section images according to an embodiment of the present disclosure;

FIGS. 2(a) to 2(d) exemplarily illustrate determination of first coordinates on the first tissue section through the fourth tissue section according to an embodiment of the present disclosure;

FIG. 3 exemplarily shows: (a) a cross-sectional image of a paraffin-embedded tissue block before slicing, (b) a slide image of the sliced tissue section before image correction, and (c) a corrected slide image; FIGS. 3(a) through 3(c) also exemplarily illustrate locations of three marker points A, B, and C on the images according to an embodiment of the present disclosure;

FIGS. 4(a) and 4(b) exemplarily illustrate two ways of dividing a quadrilateral region formed by four marker points A-D into two triangular regions according to an embodiment of the present disclosure;

FIG. 5 schematically illustrates the structural arrangement of a tissue section processing system with a film laminating mechanism in the initial state according to an embodiment of the present disclosure;

FIG. 6 schematically illustrates the structural arrangement of the tissue section processing system with the film laminating mechanism in the lamination state according to an embodiment of the present disclosure; and

FIG. 7 schematically illustrates a bottom-up view of the tissue section processing system with the film laminating mechanism in the mobile stage according to an embodiment of the present disclosure.

### DETAILED DESCRIPTION OF THE PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

In the description of the present disclosure, it should be understood that terms for expressing direction such as front, rear, left, right, up, down, vertical, horizontal, high, low, inside, outside, etc. are used to explain the orientation or position with reference to the drawings, are intended merely to simplify the description of the present disclosure, rather than indicating or implying that the device or the element must be constructed or operated in such specific orientation or position, and therefore should not be construed as limiting the scope of the present disclosure.

### Embodiment 1

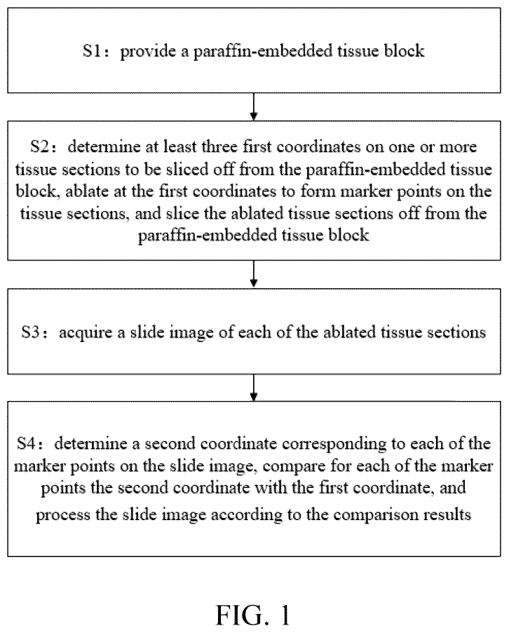

As shown in FIG. 1, a method for processing tissue section images is provided. The method includes the steps of:
(S1) providing a paraffin-embedded tissue block;
(S2) determining at least three first coordinates on one or more tissue sections to be sliced off from the paraffin-embedded tissue block, ablating at the first coordinates to form marker points on the tissue sections, and slicing the ablated tissue sections off from the paraffin-embedded tissue block;
(S3) acquiring a slide image of each of the ablated tissue sections; and
(S4) determining a second coordinate corresponding to each of the marker points on the slide image, comparing for each of the marker points the second coordinate with the first coordinate, and processing the slide image according to the comparison results.

In some embodiments, the laser is a femtosecond laser.

In one or more embodiments, the depth of the laser ablation, i.e., the length in a direction perpendicular to each cross-section of the tissue, is an integer multiple of the thickness of one tissue section, thereby improving efficiency of the ablation. In some preferred embodiments, the depth of laser ablation equals the thickness of one tissue section, so as to ensure that the marker points consistently fall within a preferred region (e.g., the tissue contour) across every tissue section.

In one or more embodiments, marker points can be randomly ablated on the cross-section of the paraffin-embedded tissue block, and the first coordinate of each marker point is identified and recorded after the marker point is ablated. In some preferred embodiments, the first coordinates of marker points on a given tissue section can be determined first, and then the laser is moved to the first coordinates to ablate the tissue section and form the marker points.

In some preferred embodiments, in order to use the affine transformation matrix to correct each slide image, the marker points ablated on the tissue section are not collinear. In some embodiments, the marker points do not form the vertices of an isosceles triangle, so as to avoid situations where the comparison results between the first and second coordinates cannot be used for rotating or flipping the slide image.

In one or more embodiments, the first coordinates of the marker points on different tissue sections can be identical or different. In one or more embodiments, the number of marker points on different tissue sections can be identical or different. In some preferred embodiments, the number of marker points on the tissue section is between 3 and 6. In some preferred embodiments, the marker points are located near the center of the tissue section.

In some preferred embodiments, the step of (S2) includes steps of: acquiring a cross-sectional image of the tissue section; extracting a tissue contour from the cross-sectional image; determining within the tissue contour the first coordinates; ablating the tissue section at the first coordinates to form the marker points thereon; and slicing the tissue section off to obtain the ablated tissue section. In some embodiments, first-order edge detection operators, such as Roberts Cross, Prewitt, Sobel, Kirsch, or compass operators, can be used to extract the tissue contour from the cross-sectional image. Additionally, in some embodiments, second-order edge detection operators, such as the Canny operator or Laplacian operator, can be used to extract the tissue contour. Preferably, the Canny operator or Sobel operator is used to extract the tissue contour from the cross-sectional image.

In some preferred embodiments, the first coordinates are determined according to the following steps: determining a centroid of the tissue contour; and generating the first coordinates around the centroid, wherein a geometric center of a polygon formed by lines connecting the first coordinates falls within a vicinity of the centroid. In the present embodiments, by locating the geometric center of the polygon formed by the marker points within the vicinity of the centroid of the tissue contour, the marker points are evenly distributed across the tissue section, thereby avoiding severe local damage to the tissue section caused by clustering of marker points.

### Embodiment 2

On the basis of Embodiment 1, the method for determining the first coordinates further includes the following steps:
for a first tissue section of the paraffin-embedded tissue block, determining a first centroid of a first tissue contour of the first tissue section, and generating the first coordinates around the first centroid, wherein a geometric center of a first polygon formed by lines connecting the first coordinates coincides with the first centroid; and
for each of the subsequent tissue sections, obtaining the first coordinates of the marker points on the preceding tissue section, shifting the first coordinates toward a same direction and for a same distance, determining whether all of the shifted first coordinates fall within a second tissue contour of the current tissue section, and
if all the shifted first coordinates fall within in the second tissue contour, defining the shifted first coordinates as the first coordinates of the marker points to be ablated on the current tissue section; or
if at least one of the shifted first coordinates fall outside of the second tissue contour, determining a second centroid of the second tissue contour, relocating the shifted first coordinates so that a geometric center of a second polygon formed by lines connecting the relocated first coordinates coincides with the second centroid, and defining coordinates corresponding to vertices of the second polygon as the first coordinates of the marker points to be ablated on the current tissue section.

In the present embodiments, the first coordinates for the subsequent tissue section are selected according to the first coordinates of the preceding tissue section, so as to ensure that the first coordinates on adjacent tissue sections do not overlap, thereby effectively reducing the likelihood of laser ablation to affect the cancerous area on the tissue section.

In one or more embodiments, the shifting distance of each first coordinate may be greater than the diameter of the cancerous region. For example, if the diameter of the cancerous region is 10 μm, the shifting distance of the first coordinates is set to be greater than 10 μm, thereby allowing the first coordinate to shift away from the cancerous region.

In some preferred embodiments, for each of the subsequent tissue sections, the first coordinates are determined according to the following step: if the first coordinates of the marker points on the preceding tissue section are shifted toward a first direction and if all of the shifted first coordinates are determined to fall within the second tissue contour, further shifting the shifted first coordinates in the first direction to generate the first coordinates of the marker points to be ablated on the current tissue section. In some preferred embodiments, after the geometric center of the polygon is moved to coincide with the centroid of the tissue contour, the shifted first coordinates are preferably shifted along a second direction that differs from the first direction.

### Embodiment 3

Embodiment 3 provides an exemplary process for determining the first coordinates for the first to fourth tissue sections to illustrate the method of determining the first coordinates for adjacent tissue sections as described in Embodiment 2.

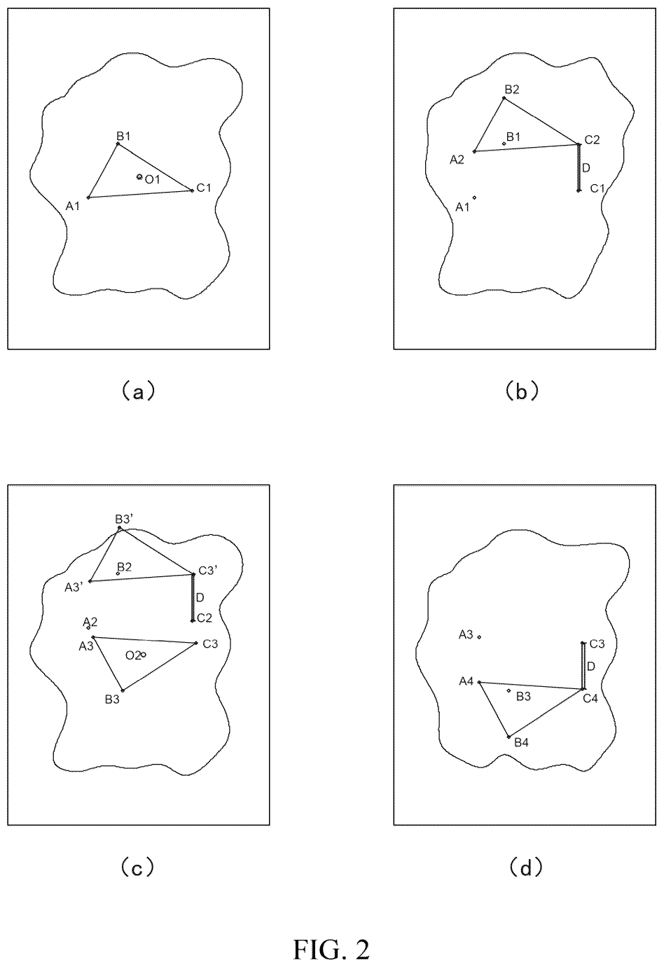

As shown in FIG. 2(a), the first tissue contour of the first tissue section is acquired, and the first centroid O1 of the first tissue contour is calculated. The first coordinates of three marker points A1, B1, and C1 are randomly determined within the first tissue contour, while a geometric center of a triangle formed by lines connecting the marker points A1, B1, and C1 coincides with the first centroid O1.

As shown in FIG. 2(b), the first coordinates of the three marker points A1, B1, and C1 are shifted upwards by a distance D to obtain shifted first coordinates located at points A2, B2, and C2. Since all of the three new points lie within the tissue contour of the second tissue section, the shifted first coordinates are defined as the first coordinates of the marker points for the second tissue section.

As shown in FIG. 2(c), the first coordinates of the three marker points A2, B2, and C2 are again shifted upwards by a distance D to obtain shifted first coordinates located at points A3′, B3′, and C3′. As exemplarily illustrated, the point B3′ lies outside the tissue contour of the third tissue section. The second tissue contour of the third tissue section is obtained and the second centroid O2 of the second tissue contour is calculated. A geometric center of a triangle formed by lines connecting the points A3′, B3′, and C3′ is moved to coincide with the second centroid O2, and the triangle is rotated by 180°. The coordinates of the vertices of the rotated triangle formed by A3, B3, and C3 are defined as the first coordinates for the third tissue section.

As shown in FIG. 2(d), the first coordinates of the three marker points A3, B3, and C3 are shifted downward by a distance D to obtain shifted first coordinates located at points A4, B4, and C4. Since all of the three new points lie within the tissue contour of the fourth tissue section, the shifted first coordinates are defined as the first coordinates of the marker points for the fourth tissue section.

The aforementioned process repeats until the first coordinates for all tissue sections are determined.

Those skilled in the art will understand that the first coordinates may be shifted in any random direction for any random distance. Preferably, the shifting distance of the first coordinates can be greater than the diameter of the cancerous region to more effectively avoid the cancerous region.

### Embodiment 4

On the basis of the aforementioned embodiments, the step of (S2) includes a step of: prior to slicing a tissue section off from the paraffin-embedded tissue block, adhering a film onto the tissue section.

In some embodiments, the thin film can be applied to the tissue section before the marker points are ablated on the tissue section. In one or more embodiments, the tissue section may be ablated the marker points first, followed by adhesion of the thin film to the ablated tissue section.

In one or more embodiments, the film may be a PEEK (polyetheretherketone) film.

### Embodiment 5

On the basis of the aforementioned embodiments, the step of (S4) further includes steps of:
for each of the marker points, comparing the first and second coordinates to obtain an affine transformation matrix;
applying the affine transformation matrix to the slide image to obtain a third coordinate for each of the marker points; and
processing the slide image according to comparison results between the third coordinates and the first coordinates.

After the second coordinates of the marker points on the slide image are determined, an affine transformation matrix corresponding to the slide image is obtained based on the first and second coordinates. The formula for calculating the affine transformation matrix is as follows:

$$[ \begin{matrix}{x}^{′} \\ {y}^{′} \\ 1\end{matrix} ] = [ \begin{matrix}{s}_{x} & {α}_{x} & {t}_{x} \\ {α}_{y} & {s}_{y} & {t}_{y} \\ 0 & 0 & 1\end{matrix} ] [ \begin{matrix}x \\ y \\ 1\end{matrix} ]$$

wherein x and y are the first coordinates (x, y), x′ and y′ are the second coordinates (x′, y′), and the six variables in the affine transformation matrix are as follows: sₓ and s_{y} respectively control the scaling and flipping along the X and Y axes, αₓ and α_{y} respectively control the rotation along the X and Y axes, and tₓ and t_{y} respectively control the translational motion along the X and Y axes.

Based on the first and second coordinates of the marker points on the tissue section and on the slide image respectively, the six variables in the affine transformation matrix can be obtained by solving a system of six linear equations. For example, the first coordinates of the three marker points formed on the tissue section are (x₁,y₁), (x₂,y₂), (x₃,y₃), and the second coordinates of the three marker points extracted from the slide image are (x₁′, y₁′), (x₂′, y₂′), (x₃′, y₃′). A system of six equations can be obtained as follows according to the formula for calculating the affine transformation matrix:

$${x}_{1}^{′} = {s}_{x} \cdot {x}_{1} + {α}_{x} \cdot {y}_{1} + {t}_{x}$$

$${y}_{1}^{′} = {α}_{y} \cdot {x}_{1} + {s}_{y} \cdot {y}_{1} + {t}_{y}$$

$${x}_{2}^{′} = {s}_{x} \cdot {x}_{2} + {α}_{x} \cdot {y}_{2} + {t}_{x}$$

$${y}_{2}^{′} = {α}_{y} \cdot {x}_{2} + {s}_{y} \cdot {y}_{2} + {t}_{y}$$

$${x}_{3}^{′} = {s}_{x} \cdot {x}_{3} + {α}_{x} \cdot {y}_{3} + {t}_{x}$$

$${y}_{3}^{′} = {α}_{y} \cdot {x}_{3} + {s}_{y} \cdot {y}_{3} + {t}_{y}$$

The six variables of the affine transformation matrix can be calculated by solving the system of six equations. Then the affine transformation matrix is applied to all pixels in the slide image to correct the slide image.

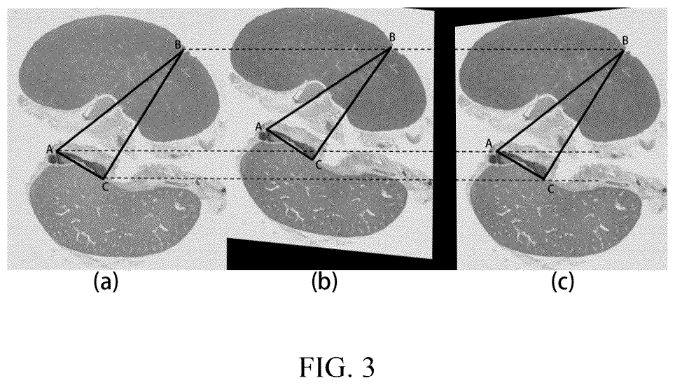

As shown in FIG. 3(a), laser ablation on the end face of the paraffin-embedded tissue block creates three marker points A, B, and C. After slicing, the tissue section is deformed, causing three marker points on the slide image to shift away to new locations, as shown in FIG. 3(b). By applying the calculated affine transformation matrix to the slide image, the slide image is corrected, as shown in FIG. 3(c). In the corrected slide image, marker points A, B, and C return to their original locations on the tissue section.

### Embodiment 6

On the basis of the aforementioned embodiments, if one or more of the tissue sections is ablated with at least four marker points, the step of (S4) includes the steps of:
dividing a polygon formed by lines connecting the at least four marker points into at least two triangular regions;
obtaining the affine transformation matrix of each triangular region; and
for pixels of the slide image locating within one of the triangular regions, processing the slide image by applying to the pixels the affine transformation matrix corresponding to the triangular region at which the pixels locate; or
for other pixels of the slide image locating outside of any one of the triangular regions, processing the slide image by applying to the other pixels the affine transformation matrix corresponding to the triangular region closest to which the other pixels locate.

In some preferred embodiments, the step of (S4) further includes the steps of: dividing the polygon in at least two ways to form at least two groups of triangular regions, and obtaining at least two groups of affine transformation matrices for each group of the triangular regions; separately processing the slide image by applying the at least two groups of the affine transformation matrices to obtain at least two corrected tissue contours of the slide image; and selecting the group of affine transformation matrices corresponding to the corrected tissue contour that has the highest resemblance to a cross-sectional image of a tissue section acquired before the tissue section is sliced off from the paraffin-embedded tissue block.

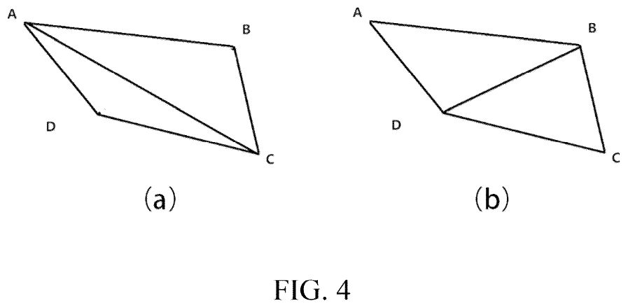

Taking a quadrilateral region formed by marker points A-D as an example, as shown in FIG. 4(a), a line connecting marker points A and C divides the quadrilateral region into two triangular regions ABC and ADC. A first affine transformation matrix is acquired based on the first and second coordinates of marker points A, B, and C in triangular region ABC. A second affine transformation matrix is acquired based on the first and second coordinates of marker points A, D, and C in triangular region ADC. The first and second affine transformation matrices are then used to correct the slide image. Specifically, a pixel in triangular region ABC is corrected by the first affine transformation matrix, while a pixel in triangular region ADC is corrected by the second affine transformation matrix. For a pixel outside the triangular region ABC or ADC, the distance between the pixel and the two triangular regions is calculated and compared, and the pixel is corrected by the affine transformation matrix corresponding to the triangular region closer to the pixel. After correction, a first corrected slide image is obtained, and the tissue contour of the first corrected slide image is extracted.

As shown in FIG. 4(b), a line connecting marker points B and D divides the quadrilateral region into two triangular regions ABD and BCD. A third affine transformation matrix is acquired based on the first and second coordinates of marker points A, B, and D in triangular region ABD. A fourth affine transformation matrix is acquired based on the first and second coordinates of marker points B, C, and D in triangular region BCD. The third and fourth affine transformation matrices are then used to correct the slide image. Specifically, a pixel in triangular region ABD is corrected by the third affine transformation matrix, while a pixel in triangular region BCD is corrected by the fourth affine transformation matrix. For a pixel outside the triangular region ABD or BCD, the distance between the pixel and the two triangular regions is calculated and compared, and the pixel is corrected by the affine transformation matrix corresponding to the triangular region closer to the pixel. After correction, a second corrected slide image is obtained, and the tissue contour of the second corrected slide image is extracted.

Lastly, the tissue contours of the first and second corrected slide image are compared to the tissue contour of the cross-sectional image, and the corrected slide image whose tissue contour more closely matches the tissue contour of the cross-sectional image is chosen as the final corrected slide image. That is to say, the final corrected slide image was subjected to application of the group of affine transformation matrices that correspond to the corrected tissue contour that has the highest resemblance to the cross-sectional image of the tissue section.

### Embodiment 7

On the basis of the aforementioned embodiments, a system for processing tissue section images is also provided. The system includes:
a microtome for slicing a paraffin-embedded tissue block to obtain a plurality of tissue sections;
a computing device for determining at least three first coordinates on at least a portion of the tissue sections;
a laser for emitting laser beams to ablate at least three marker points at the first coordinates on each of the tissue sections;
a tissue scanner for acquiring a slide image of each of the ablated tissue sections; and
an image processor for determining a second coordinate corresponding to each of the marker points on the slide image, comparing for each of the marker points the second coordinate with the first coordinate, and processing the slide image according to the comparison results.

### Embodiment 8

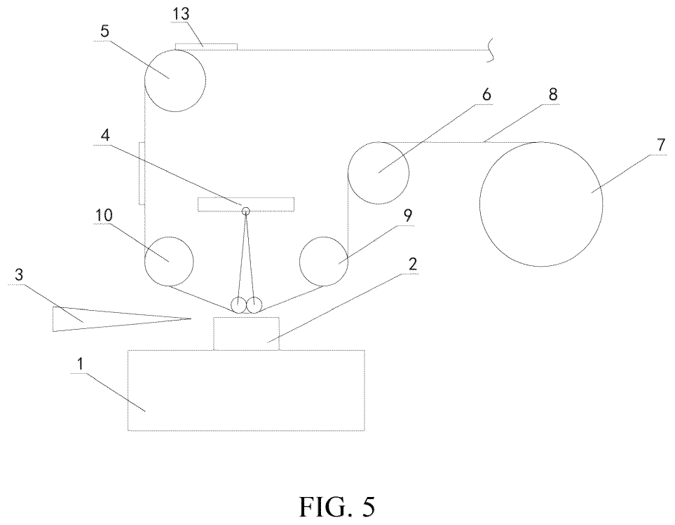

On the basis of the aforementioned embodiments, as shown in FIG. 5 to FIG. 7, a system for processing tissue section images includes a conveyor mechanism for driving movement of a film 8. Along a movement path of the film 8, the system further includes a sample placement stage 1 for placing a paraffin-embedded tissue block 2, a blade 3 of the microtome for slicing the paraffin-embedded tissue block 2, a film laminating mechanism 4 disposed directly above the paraffin-embedded tissue block 2, and a laser 16 mounted on the film laminating mechanism 4.

When in use, the film 8 is coated with an adhesive, such as formaldehyde gelatin solution or polylysine solution. When the film 8 moves to above the paraffin-embedded tissue block 2, the conveyor mechanism stops driving the film 8. The film laminating mechanism 4 moves downward to attach the film 8 to a surface of the paraffin-embedded tissue block 2. The laser 16 emits a laser beam to ablate marker points on the paraffin-embedded tissue block 2. The blade 3 slices the ablated tissue section off from the paraffin-embedded tissue block 2; thus, the sliced tissue section is adhered to and captured by the film 8. As the film 8 is driven by the conveyor mechanism to move again, the sliced tissue section moves away from the sample placement stage 1 with the film 8 for subsequent processing. Such process is repeated until all tissue sections are captured.

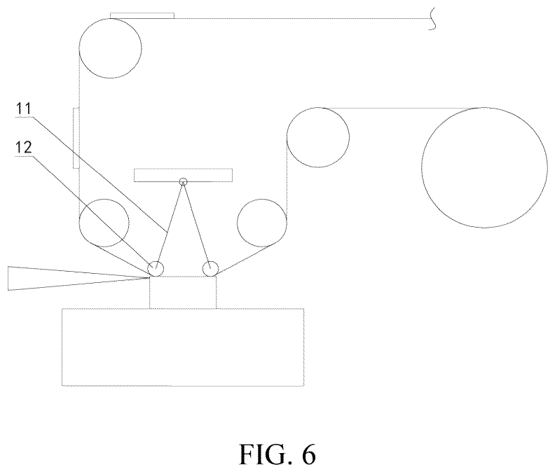

In some preferred embodiments, as shown in FIG. 5 and FIG. 6, the conveyor mechanism includes a reel 7 wound with the film 8 and a driving device for pulling the film 8 out from the reel 7. In one or more embodiments, an independently motor-driven first roller 6 and an independently motor-driven second roller 5 are disposed on the movement path of the film 8. In one or more embodiments, a third roller 9 and a fourth roller 10 disposed between the first roller 6 and the second roller 5 are respectively disposed on each side of the film laminating mechanism 4 and independently driven by a motor as well.

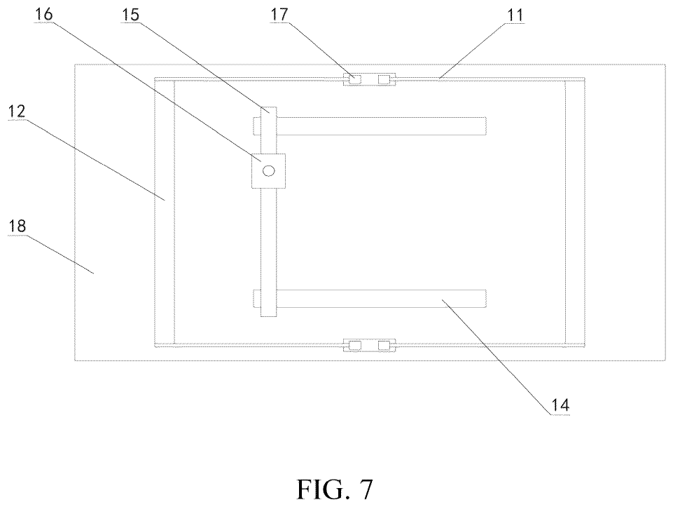

In some preferred embodiments, as shown in FIG. 7, the film laminating mechanism 4 includes a movable platform 18 capable of moving vertically. A horizontal linear guide rail pair 14 is disposed on a lower surface of the movable platform 18. A vertical linear guide rail pair 15 is slidably arranged on the horizontal linear guide rail pair 14, and the laser 16 is slidably arranged on the vertical linear guide rail pair 15. Before marker points are ablated, the sliders of the horizontal and vertical linear guide rail pairs are moved according to the determined first coordinates, thereby moving the laser 16 to the required position for ablation.

In some preferred embodiments, as shown in FIG. 7, the lower surface of the movable platform 18 is hinge-connected to a connecting rod 11 via a bearing 17, and a torsion spring is disposed at the hinge connection. A film application roller 12 is rotatably connected to the connecting rod 11. In one or more embodiments, two film application rollers 12 are hinged to the lower surface of the movable platform 18.

At the initial state, as shown in FIG. 5, two film application rollers 12 are located directly above the paraffin-embedded tissue block 2 and abut against each other under the action of the torsion spring. During the downward movement of the film laminating mechanism 4, the film application rollers 12 push the film 8 toward the paraffin-embedded tissue block 2. At the lamination state, as shown in FIG. 6, the film 8 contacts the paraffin-embedded tissue block 2, and the film application rollers move from the center of the paraffin-embedded tissue block 2 to two end sides thereof, thereby expelling air between the film 8 and the paraffin-embedded tissue block 2. After film lamination is completed, the film laminating mechanism 4 resets upward, and the two film application rollers 12 return to the initial state as shown in FIG. 5 under the action of the torsion spring.

Terms such as “first”, “second” (e.g., first coordinate, second coordinate; first centroid, second centroid) used herein are merely distinguish the components for clear description, and do not intend to limit any sequence or emphasize the importance. In addition, the term “connect” used herein may refer to direct connection or indirect connection via other components.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A method for processing tissue section images, comprising steps of:
(S1) providing a paraffin-embedded tissue block;
(S2) determining at least three first coordinates on one or more tissue sections to be sliced off from the paraffin-embedded tissue block, ablating at the first coordinates to form marker points on the tissue sections, and slicing the ablated tissue sections off from the paraffin-embedded tissue block;
(S3) acquiring a slide image of each of the ablated tissue sections; and
(S4) determining a second coordinate corresponding to each of the marker points on the slide image, comparing for each of the marker points the second coordinate with the first coordinate, and processing the slide image according to the comparison results.

2. The method of claim 1, wherein the step of (S2) comprises steps of:
acquiring a cross-sectional image of the tissue section;
extracting a tissue contour from the cross-sectional image;
determining within the tissue contour the first coordinates;
ablating the tissue section at the first coordinates to form the marker points thereon; and
slicing the tissue section off to obtain the ablated tissue section.

3. The method of claim 2, wherein the first coordinates are determined according to the following steps:
determining a centroid of the tissue contour; and
generating the first coordinates around the centroid, wherein a geometric center of a polygon formed by lines connecting the first coordinates falls within a vicinity of the centroid.

4. The method of claim 2, wherein the first coordinates are determined according to the following steps:
for a first tissue section of the paraffin-embedded tissue block, determining a first centroid of a first tissue contour of the first tissue section, and generating the first coordinates around the first centroid, wherein a geometric center of a first polygon formed by lines connecting the first coordinates coincides with the first centroid; and
for each of the subsequent tissue sections, obtaining the first coordinates of the marker points on the preceding tissue section, shifting the first coordinates toward a same direction and for a same distance, determining whether all of the shifted first coordinates fall within a second tissue contour of the current tissue section, and
if all the shifted first coordinates fall within in the second tissue contour, defining the shifted first coordinates as the first coordinates of the marker points to be ablated on the current tissue section; or
if at least one of the shifted first coordinates fall outside of the second tissue contour, determining a second centroid of the second tissue contour, relocating the shifted first coordinates so that a geometric center of a second polygon formed by lines connecting the relocated first coordinates coincides with the second centroid, and defining coordinates corresponding to vertices of the second polygon as the first coordinates of the marker points to be ablated on the current tissue section.

5. The method of claim 4, wherein if at least one of the shifted first coordinates fall outside of the second tissue contour, the method further comprises a step of:
after relocating the shifted first coordinates, rotating the second polygon formed by the lines connecting the relocated first coordinates.

6. The method of claim 4, wherein for each of the subsequent tissue sections, the first coordinates are determined according to the following step:
if the first coordinates of the marker points on the preceding tissue section are shifted toward a first direction and if all of the shifted first coordinates are determined to fall within the second tissue contour, further shifting the shifted first coordinates in the first direction to generate the first coordinates of the marker points to be ablated on the current tissue section.

7. The method of claim 1, wherein the step of (S2) comprises a step of:
prior to slicing a tissue section off from the paraffin-embedded tissue block, adhering a film onto the tissue section.

8. The method of claim 1, wherein the step of (S4) comprises steps of:
for each of the marker points, comparing the first and second coordinates to obtain an affine transformation matrix;
applying the affine transformation matrix to the slide image to obtain a third coordinate for each of the marker points;
processing the slide image according to comparison results between the third coordinates and the first coordinates.

9. The method of claim 8, wherein if one or more of the tissue sections is ablated with at least four marker points, the step of (S4) comprises steps of:
dividing a polygon formed by lines connecting the at least four marker points into at least two triangular regions;
obtaining the affine transformation matrix of each triangular region; and
for pixels of the slide image locating within one of the triangular regions, processing the slide image by applying to the pixels the affine transformation matrix corresponding to the triangular region at which the pixels locate; or
for other pixels of the slide image locating outside of any one of the triangular regions, processing the slide image by applying to the other pixels the affine transformation matrix corresponding to the triangular region closest to which the other pixels locate.

10. The method of claim 9, wherein the step of (S4) further comprises steps of:
dividing the polygon in at least two ways to form at least two groups of triangular regions, and obtaining at least two groups of affine transformation matrixes for each group of the triangular regions;
separately processing the slide image by applying the at least two groups of the affine transformation matrixes to obtain at least two corrected tissue contours of the slide image; and
selecting the group of affine transformation matrixes corresponding to the corrected tissue contour that has the highest resemblance to a cross-sectional image of a tissue section acquired before the tissue section is sliced off from the paraffin-embedded tissue block.

11. A system for processing tissue section images, comprising:
a microtome for slicing a paraffin-embedded tissue block to obtain a plurality of tissue sections;
a computing device for determining at least three first coordinates on at least a portion of the tissue sections;
a laser for emitting laser beams to ablate at least three marker points at the first coordinates on each of the tissue sections;
a tissue scanner for acquiring a slide image of each of the ablated tissue sections; and
an image processor for determining a second coordinate corresponding to each of the marker points on the slide image, comparing for each of the marker points the second coordinate with the first coordinate, and processing the slide image according to the comparison results.

12. The system of claim 11, wherein the computing device is further controlled for acquiring a cross-sectional image of the tissue section, extracting a tissue contour from the cross-sectional image, and determining within the tissue contour the first coordinates.

13. The system of claim 12, wherein the computing device is controlled to determine the first coordinates according to the following steps:
determining a centroid of the tissue contour; and
generating the first coordinates around the centroid, wherein a geometric center of a polygon formed by lines connecting the first coordinates falls within a vicinity of the centroid.

14. The system of claim 12, wherein the computing device is further controlled to determine the first coordinates according to the following steps:
for a first tissue section of the paraffin-embedded tissue block, determining a first centroid of a first tissue contour of the first tissue section, and generating the first coordinates around the first centroid, wherein a geometric center of a first polygon formed by lines connecting the first coordinates coincides with the first centroid; and
for each of the subsequent tissue sections, obtaining the first coordinates of the marker points on the preceding tissue section, shifting the first coordinates toward a same direction and for a same distance, determining whether all of the shifted first coordinates fall within a second tissue contour of the current tissue section, and
if all the shifted first coordinates fall within in the second tissue contour, defining the shifted first coordinates as the first coordinates of the marker points to be ablated on the current tissue section; or
if at least one of the shifted first coordinates fall outside of the second tissue contour, determining a second centroid of the second tissue contour, relocating the shifted first coordinates so that a geometric center of a second polygon formed by lines connecting the relocated first coordinates coincides with the second centroid, and defining coordinates corresponding to vertices of the second polygon as the first coordinates of the marker points to be ablated on the current tissue section.

15. The system of claim 14, wherein if at least one of the shifted first coordinates fall outside of the second tissue contour, the computing device is further controlled to perform the following step:
after relocating the shifted first coordinates, rotating the second polygon formed by the lines connecting the relocated first coordinates.

16. The system of claim 14, wherein for each of the subsequent tissue sections, the computing device is further controlled to determine the first coordinates according to the following step:
if the first coordinates of the marker points on the preceding tissue section are shifted toward a first direction and if all of the shifted first coordinates are determined to fall within the second tissue contour, further shifting the shifted first coordinates in the first direction to generate the first coordinates of the marker points to be ablated on the current tissue section.

17. The system of claim 11, further comprising a film adhering device for adhering a film onto the tissue sections before the tissue sections are sliced off from the paraffin-embedded tissue block.

18. The system of claim 11, wherein the image processor is further controlled to perform the following steps:
comparing, for each of the marker points, the first and second coordinates to obtain an affine transformation matrix;
applying the affine transformation matrix to the slide image to obtain a third coordinate for each of the marker points; and
processing the slide image according to comparison results between the third coordinates and the first coordinates.

19. The system of claim 18, wherein the image processor is further controlled to perform the following steps:
if one or more of the tissue sections is ablated with at least four marker points, dividing a polygon formed by lines connecting the at least four marker points into at least two triangular regions;
obtaining the affine transformation matrix of each triangular region; and
for pixels of the slide image locating within one of the triangular regions, processing the slide image by applying to the pixels the affine transformation matrix corresponding to the triangular region at which the pixels locate; or
for other pixels of the slide image locating outside of any one of the triangular regions, processing the slide image by applying to the other pixels the affine transformation matrix corresponding to the triangular region closest to which the other pixels locate.

20. The system of claim 19, wherein the image processor is further controlled to perform the following steps:
dividing the polygon in at least two ways to form at least two groups of triangular regions, and obtaining at least two groups of affine transformation matrixes for each group of the triangular regions;
separately processing the slide image by applying the at least two groups of the affine transformation matrixes to obtain at least two corrected tissue contours of the slide image; and
selecting the group of affine transformation matrixes corresponding to the corrected tissue contour that has the highest resemblance to a cross-sectional image of a tissue section acquired before the tissue section is sliced off from the paraffin-embedded tissue block.
